(19) United States
(12) Patent Application Publication
(10) Pub. No.: US 2026/0001060 A1
(43) Pub. Date: Jan. 1, 2026

(54) HYDRODEOXYGENATION CATALYST AND PREPARATION METHOD THEREOF

(71) Applicant: THE BOEING COMPANY, Chicago, IL (US)

(72) Inventors: Yong Guo, Shanghai (CN); Yanqin Wang, Shanghai (CN); Zhoa Wang, Beijing (CA)

(21) Appl. No.: 18/881,894

(22) PCT Filed: Jul. 20, 2023
(86) PCT No.: PCT/US23/70554

§ 371 Date: Jan. 7, 2025

(30) Foreign Application Priority Data
Jul. 20, 2022 (CN) 202210858407.2

(51) Int. Cl. B01J 23/648; B01J 37/02; B01J 37/03; B01J 37/04; B01J 37/08; B01J 37/18; C07C 1/247

(52) U.S. Cl. CPC B01J 23/6484; B01J 37/0236; B01J 37/036; B01J 37/04; B01J 37/088; B01J 37/18; C07C 1/247; C07C 2523/20

(57) ABSTRACT

The present disclosure relates to a hydrodeoxygenation catalyst and a preparation method thereof. Specifically, the hydrodeoxygenation catalyst contains a hydrogenation active component and a catalyst carrier, the hydrogenation active component contains one or more hydrogenation active metals that comprise Pt, Pd, Rh, Ru, Ni, Co, Cu, or a combination thereof, and the catalyst carrier is a solid solution composite oxide containing A1, Nb, Si, and O elements, and the catalyst carrier is represented by a chemical formula (Nb₂O₅)ₓ·(Al₂O₃)_{y}·(SiO₂)_{z}, wherein 0.01≤x≤0.3, 0.01≤y≤0.1 and 0.6≤z≤0.98.

### CROSS REFERENCE TO RELATED APPLICATIONS

The present application claims priority from Patent Cooperation Treaty Application PCT/US2023/070554, filed on Jul. 20, 2023, and titled “Hydrodeoxygenation Catalyst and Preparation Method Thereof,” which claims priority to Chinese Patent Application 202210858407.2, filed on Jul. 20, 2022, each of which is incorporated herein by reference in its entirety.

### TECHNICAL FIELD

The present disclosure relates to the technical field of biological energy, in particular to a hydrodeoxygenation catalyst and a preparation method thereof, and a method for preparing bio-jet fuel component oil using the hydrodeoxygenation catalyst.

### BACKGROUND

In the background of the desire to reduce reliance on petroleum-based fuels and mitigate greenhouse gases, a biomass is currently the most promising resource that may be used to prepare a hydrocarbon liquid fuel to replace petroleum derived fuel. In order to continue the transition from a fossil energy economy to a renewable energy economy and convert the biomass into high value-added fuels and chemicals, furfural, a lignocellulosichydrolyzate, is used as a raw material, and firstly extends a carbon chain by an aldol condensation reaction to obtain furfurylacetone (4-(2-furyl)-3-buten-2-one) and difurfurylacetone (1,5-bis-(2-furyl)-1,4-pentadiene-3-one), then hydrodeoxygenates to obtain a C₈ straight-chain alkane and a C₁₃ straight-chain alkane, and finally isomerizes to obtain a qualified jet fuel component, as to achieve the efficient utilization of the biomass (Science, 308, 1446-1450 (2005)).

The research on a Claisen condensation reaction of furfural and acetone catalyzed by an inorganic strong alkali to prepare difurfurylacetone (1,5-bis-(2-furyl)-1,4-pentadien-3-one) is relatively mature already, and a waste liquid treatment problem is already solved by a mild preparation process previously. The difurfurylacetone obtained by the reaction may not only be used as an intermediate of the jet fuel to obtain a C₁₃ hydrocarbon component by hydrodeoxygenation, as to be added to the jet fuel for use; but may also be used as a high value-added chemical which is usable as a co-crosslinking agent and a co-vulcanizing agent for rubber, as to improve performances such as the co-crosslinking degree, the heat resistance and the tensile stress of the rubber.

It is difficult to directly perform hydrodehydration on the difurfurylacetone using an existing hydrodehydration catalyst. An existing method adopted is to pre-hydrogenate a carbon-carbon double bond to obtain 1,5-bis(2-tetrahydrofuranyl)-3-pentanone, and then use the hydrodehydration catalyst to dehydrate and hydrogenate it to obtain a C₁₃ long-chain alkane. Such a step-by-step hydrogenation method (two-step hydrogenation) may greatly reduce the difficulty of preparing the jet fuel component. In addition, the step-by-step hydrogenation method may also improve the yield of target long-chain alkanes. In view of these advantages, most of existing long-chain alkane preparation methods adopted in existing technologies is the step-by-step hydrogenation method, and a reaction route of the difurfurylacetone to generate bio-jet fuel component oil by the two-step hydrogenation is as follows:

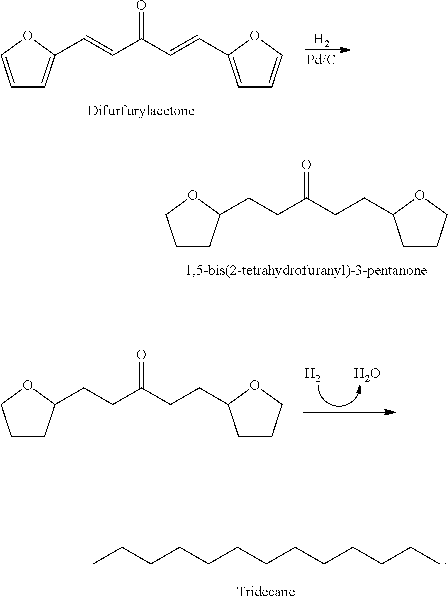

However, the existing catalyst for catalyzing the hydrodehydration of the difurfurylacetone in the existing technology only supports the preparation of the long-chain alkane by the above step-by-step hydrogenation method, and there is a deficiency in catalytic efficiency, and an improved production method for a biomass-based jet fuel component is required.

Furthermore, it is known that the bottleneck in the production of the biomass-based jet fuel component lies in the development of inexpensive and efficient hydrodeoxygenation catalysts. Therefore, there is still a need for the inexpensive and efficient hydrodeoxygenation catalysts in this field.

### SUMMARY

A purpose of the present disclosure is to provide a hydrodeoxygenation catalyst and a preparation method thereof, and a method for preparing bio-jet fuel component oil using the hydrodeoxygenation catalyst, as to solve problem of existing hydrodeoxygenation catalysts for catalyzing biomass hydrodeoxygenation to prepare bio-jet fuel component oil, such as low catalytic efficiency, low conversion rate of raw materials, low product yield and poor selectivity.

In order to achieve the above purpose, according to one aspect of the present disclosure, a hydrodeoxygenation catalyst is provided, the hydrodeoxygenation catalyst includes a hydrogenation active component and a catalyst carrier, the hydrogenation active component includes one or more hydrogenation active metals, and the catalyst carrier is a solid solution composite oxide containing Nb, Al, Si, and O elements, and the catalyst carrier is represented by a chemical formula (Nb₂O₅)ₓ·(Al₂O₃)_{y}·(SiO₂)_{z}, herein 0.01≤x≤0.3, 0.01≤y≤0.1 and 0.6≤z≤0.98.

Further, the one or more hydrogenation active metals comprise Pt, Pd, Rh, Ru, Ni, Co, Cu, or a combination thereof.

Further, based on the total weight of the hydrodeoxygenation catalyst, the loading amount of the hydrogenation active metal is between about 0.4 wt % to about 10 wt %.

Further, Nb₂O₅, Al₂O₃, and SiO₂ in the hydrodeoxygenation catalyst exist in an amorphous form.

According to another aspect of the present disclosure, a preparation method for a hydrodeoxygenation catalyst is provided, and the method includes: a solation step that includes mixing a hydrogenation active metal source with a niobium source, an aluminum source and a silicon source of which the molar ratio is (0.01-0.3):(0.01-0.1):(0.6-0.98) and dissolving in water to obtain a sol of a soluble precursor; a gelation step that includes stirring the sol at a temperature within a temperature range from room temperature to about 60° C. to obtain a gel; and an aging step that includes allowing the gel to stand for aging to obtain an aged material; and drying, calcining and reducing the aged material to obtain the hydrodeoxygenation catalyst.

Further, in the solation step, the mixing of the niobium source, the aluminum source, the silicon source and the hydrogenation active metal source is performed in the presence of a hydrolyzing agent.

Further, in the gelation step, a stirring time is between about an hour and about 12 hours.

Further, in the aging step, the aging time is between about an hour and about 3 hours.

Further, the reduction is performed at a temperature in a range between about 200° C. and about 500° C., and in the presence of hydrogen for between about 2 hours and about 6 hours.

Further, the niobium source is selected from the group consisting of a niobium tartrate, a niobium citrate, a niobium malate, a niobium nitrate, a niobium hydrochloride, a niobium sulfate and a combination thereof, and preferably the niobium source is the niobium tartrate.

Further, the hydrolyzing agent is selected from the group consisting of an acid and an alkali; the aluminum source is selected from the group consisting of an aluminum nitrate, an aluminum chloride, an aluminum acetate and a combination thereof, and preferably the aluminum source is the aluminum nitrate; the silicon source is selected from the group consisting of a silica sol, water glass, an ethyl orthosilicate and a combination thereof, and preferably the silicon source is the silica sol; and the hydrogenation active metal source is selected from the group consisting of a nitrate, a sulfate, a chloride, an acetate and a combination thereof, and preferably the hydrogenation active metal source is selected from the group consisting of a palladium nitrate, a palladium sulfate, a palladium chloride, a palladium acetate, a platinum nitrate, a platinum sulfate, a platinum chloride, a platinum acetate, a ruthenium nitrate, a nickel nitrate, a cobalt nitrate, and a combination thereof.

According to yet another aspect of the present disclosure, a method for preparing an isoalkane using a hydrodeoxygenation catalyst is provided, and the method includes: in the presence of the hydrodeoxygenation catalyst and an organic solvent, performing a hydrogenation reaction on the difurfurylacetone of the following formula to obtain the isoalkane:

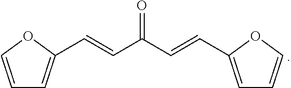 [SMILES: O=C(/C=C/c1ccco1)/C=C/c1ccco1]

Further, the hydrodeoxygenation reaction is performed at a temperature in a range between about 150° C. and about 280° C., under a hydrogen pressure in a range between about 0.5 MPa and about 8 MPa, and under stirring for between about an hour to 36 hours.

Further, the organic solvent is a cyclohexane, and based on a total weight of the difurfurylacetone and the organic solvent, the weight percent of the difurfurylacetone is between about 10 wt % to 30 wt %.

Further, a weight ratio of the difurfurylacetone to the hydrodeoxygenation catalyst is from 2:1 to 20:1.

According to a technical scheme of the present disclosure, a hydrodeoxygenation catalyst, a preparation method thereof, and a method for preparing bio-jet fuel component oil by using the hydrodeoxygenation catalyst are provided. The hydrodeoxygenation catalyst of the present disclosure adopts a catalyst carrier represented by a chemical formula (Nb₂O₅)ₓ·(Al₂O₃)_{y}·(SiO₂)_{z}, wherein 0.01≤x≤0.3, 0.01≤y≤0.1 and 0.6≤z≤0.98, the problems of the existing hydrodeoxygenation catalysts for catalyzing biomass hydrodeoxygenation to prepare bio-jet fuel component oil, such as low catalytic efficiency, low conversion rate of raw materials, low product yield and poor selectivity are solved. In the present disclosure, the above problems are solved by adopting the hydrodeoxygenation catalyst which includes the catalyst carrier of specific chemical composition of the present disclosure. Therefore, the conversion rate of raw materials, product yield and selectivity of the hydrodeoxygenation catalyst are improved.

### DETAILED DESCRIPTION

It should be noted that examples in the present application and features of the examples may be combined with each other in the case without conflicting. The present disclosure is described in detail below with reference to the examples.

In an existing technology, a hydrodeoxygenation product prepared by catalyzing biomass hydrodeoxygenation is an n-alkane, and its freezing point is relatively high. An ideal component of bio-jet fuel component oil is an isoalkane, this is because the freezing point of the isoalkane is lower than that of the n-alkane (for example, the freezing point of an isotridecane is about −30° C.). In addition, the hydrodeoxygenation catalyst for catalyzing biomass hydrodeoxygenation to prepare the bio-jet fuel component oil in the existing technology has problems of low catalytic efficiency, low conversion rate of raw materials, low product yield and poor selectivity. In view of the mentioned deficiencies in the existing technology, the present application provides a hydrodeoxygenation catalyst, the hydrodeoxygenation catalyst contains a hydrogenation active component and a catalyst carrier, the hydrogenation active component contains one or more hydrogenation active metals, and the catalyst carrier is a solid solution composite oxide containing Al, Nb, Si and O elements, and the catalyst carrier is represented by a chemical formula (Nb₂O₅)ₓ·(Al₂O₃)_{y}·(SiO₂)_{z}, herein 0.01≤x≤0.3, 0.01≤y≤0.1 and 0.6≤z≤0.98.

According to the present disclosure, the hydrodeoxygenation catalyst for catalyzing the biomass hydrodeoxygenation to prepare the long-chain alkane in the existing technology is low in efficiency, excessive in consumption of raw materials, low in conversion efficiency of raw materials and poor in product selectivity. Based on the above problems, the present disclosure prepares a hydrodeoxygenation catalyst for catalyzing hydrogenation of a difurfurylacetone to prepare bio-jet fuel component oil, and the specific catalyst uses a catalyst carrier (represented by the chemical formula (Nb₂O₅)ₓ·(Al₂O₃)_{y}·(SiO₂)_{z}, wherein 0.01≤x≤0.3, 0.01≤y≤0.1 and 0.6≤z≤0.98) including the specific chemical composition of the present disclosure to solve the above problems. The catalyst carrier of the present disclosure is a Nb₂O₅—Al₂O₃—SiO₂ ternary composite material with a specific ratio, and a Nb₂O₅ part in the composite material has the excellent C—O bond activation ability, and may efficiently catalyze a hydrodeoxygenation reaction of the difurfurylacetone to obtain the alkane, at the same time, it may also provide an acid site, to catalyze the alkane generated by the hydrodeoxygenation reaction to perform an isomerization reaction, thereby an isoalkane product is obtained; Al₂O₃ and SiO₂ parts in the composite material are used as components of a molecular sieve, which may provide the acid site, to promote the alkane generated by the hydrodeoxygenation reaction to perform the isomerization reaction. Further, x, y, and z in the catalyst carrier of the chemical formula (Nb₂O₅)ₓ·(Al₂O₃)_{y}·(SiO₂)_{z} are within a scope of the present disclosure, which may ensure that each part in the composite material effectively exerts its catalytic function, improve the conversion rate of bio-fuel oxygen-containing compound raw materials such as the difurfurylacetone in the hydrodeoxygenation reaction, and improve the yield of a C₁₀₊ long-chain alkane (such as a C₁₃ alkane), and the selectivity of the isoalkane (a main component of a sustainable aviation fuel (SAF)) is excellent.

In addition, the hydrodeoxygenation catalyst of the present disclosure includes a hydrogenation active component, and the hydrogenation active component contains a metal as a hydrogenation active material, and the hydrogenation active metal may catalyze a hydrogen molecule adsorbed on the catalyst to form a hydrogen atom, as to participate in an addition reaction with a carbon-oxygen double bond in the difurfurylacetone, to ensure the hydrogenation reaction of the difurfurylacetone.

Preferably, x is 0.01, 0.02, 0.03, 0.04, 0.05, 0.06, 0.07, 0.08, 0.09, 0.1, 0.15, 0.2, 0.25, 0.28, 0.3 or any values between them. y is 0.01, 0.02, 0.03, 0.04, 0.05, 0.06, 0.07, 0.08, 0.09, 0.1, or any values between them. z is 0.61, 0.65, 0.7, 0.75, 0.8, 0.85, 0.9, 0.95, 0.98, or any values between them. While the values of x, y, and z are within the scope of the present disclosure, Nb₂O₅ in the composite material may provide the excellent carbon-oxygen bond activation ability and the sufficient acid sites, and the Al₂O₃ and SiO₂ parts, as the components of the molecular sieve, further provide the acid sites, thereby the conversion rate of the difurfurylacetone and the C₁₀₊ long-chain alkane yield and the selectivity of the isoalkane are improved better.

Preferably, the one or more hydrogenation active metals comprise Pt, Pd, Rh, Ru, Ni, Co, Cu, or a combination thereof. More preferably, the one or more hydrogenation active metals are Pt, Pd, or a combination of Pt and Pd. The hydrogenation active metal within the scope of the present disclosure may promote the adsorption of the hydrogen molecule, thereby the conversion of the hydrogen molecule to the hydrogen atom is promoted, and the hydrogenation reaction of the difurfurylacetone is further promoted.

Preferably, the ratio between x, y and z in the chemical formula of the catalyst carrier is (0.01-0.3):(0.01-0.1):(0.6-0.98). For example, the ratio between x, y and z is 0.01:0.01:0.6, 0.05:0.01:0.6, 0.08:0.01:0.6, 0.1:0.01:0.6, 0.15:0.01:0.6, 0.2:0.01:0.6, 0.25:0.01:0.6, 0.3:0.01:0.6, 0.01:0.03:0.6, 0.01:0.05:0.6, 0.01:0.07:0.6, 0.01:0.1:0.6, 0.01:0.05:0.65, 0.01:0.05:0.7, 0.01:0.05:0.75, 0.01:0.05:0.8, 0.01:0.05:0.85, 0.01:0.05:0.9, 0.01:0.05:0.95, 0.01:0.05:0.98 and the like. According to the present disclosure, while the ratio between x, y and z is within the scope of the present disclosure, it may further provide the excellent carbon-oxygen bond activation ability and more acid sites, thereby the conversion rate of the difurfurylacetone and the yield of the C₁₀₊ long-chain alkane are further improved, and the selectivity of the isoalkane may be further improved.

In some examples, based on the total weight of the hydrodeoxygenation catalyst, the loading amount of the hydrogenation active metal is between about 0.4 wt %-10 wt %. For example, the loading amount of the hydrogenation active metal is 0.4 wt %, 0.42 wt %, 0.44 wt %, 0.46 wt %, 0.48 wt %, 0.5 wt %, 0.8 wt %, 1 wt %, 2 wt %, 3 wt %, 4 wt %, 5 wt %, 7 wt %, 8 wt %, 10 wt % and the like. If the loading amount is too high, the cost may be increased, and if the loading amount is too low, there are fewer active metal catalytic centers, and it is not beneficial to exert the hydrogenation catalytic activity. Compared with an existing hydrodeoxygenation catalyst, the catalyst of the present disclosure is lower in loading amount of an active metal component (precious metal), and better in catalytic performance, so the catalyst with excellent performance may be prepared at a lower cost.

In some examples, Nb₂O₅, Al₂O₃, and SiO₂ in the hydrodeoxygenation catalyst exist in an amorphous form. The hydrodeoxygenation catalyst synthesized by the present disclosure is a solid solution composite oxide, wherein Nb₂O₅, Al₂O₃, and SiO₂ exist in an amorphous form, and compared with the existing hydrodeoxygenation catalyst, it has more unsaturated coordination active centers, which may further improve the catalytic activation of the carbon-oxygen bond, and promote the breakage of the carbon-oxygen bond, thereby the catalytic activity of the catalyst is improved, and the conversion rate of reactants, the yield of the C₁₀₊ long-chain alkane and the selectivity of the isoalkane are further improved.

In some examples, a preparation method for a hydrodeoxygenation catalyst includes: a solation step: mixing a hydrogenation active metal source with a niobium source, an aluminum source and a silicon source of which the molar ratio (0.01-0.3):(0.01-0.1):(0.6-0.98) and dissolving in water to obtain a sol of a soluble precursor; a gelation step: stirring the sol at a temperature from a room temperature to about 60° C. to obtain a gel; and an aging step: standing the gel for aging to obtain an aged material; and drying, calcining and reducing the aged material to obtain the hydrodeoxygenation catalyst. According to the method of the present disclosure, in the solation step, the hydrogenation active metal source is mixed with the specific molar ratio of the niobium source, the aluminum source and the silicon source and dissolved in the water at a conventional temperature, the niobium source, the aluminum source and the silicon source generate a hydrolysis reaction in the water, as to form a stable and transparent sol system. In the gelation step, the sol system is stirred at the temperature from the room temperature to about 60° C., and a slow cross-linking reaction occurs between colloidal particles of the sol to form a gel system with a three-dimensional network structure. In the aging step, the aging occurs while the gel system is left standing for a period of time, and the aging process makes the cross-linking of the gel more sufficient and the three-dimensional network structure more stable. The aged material is dried, and moisture in it is removed by volatilization; the calcinating process causes each component in the dried aged material to generate a decomposition reaction; then the calcinated aged material is reduced, so that the hydrogenation active metal in the form of the oxide is reduced and an oxygen vacancy is created, and finally the metal active center with activated hydrogen, and the oxygen vacancy center that improves the C—O bond activation ability are obtained. Thus, the catalyst carrier with the specific chemical composition of the present disclosure is obtained (represented by the chemical formula (Nb₂O₅)ₓ·(Al₂O₃)_{y}·(SiO₂)_{z}, wherein 0.01≤x≤0.3, 0.01≤y≤0.1 and 0.6≤z≤0.98).

In some examples, in the solation step, the mixing of the niobium source, the aluminum source, the silicon source, and the hydrogenation active metal source is performed in the presence of a hydrolyzing agent. The presence of the hydrolyzing agent may further promote the hydrolysis process of the aluminum source, the niobium source and the silicon source, and thereby, the formation of the sol system is further promoted. On the other hand, the addition of the hydrolyzing agent may promote the polymerization reaction process between the colloidal particles of the sol and the formation of the three-dimensional network structure, which is beneficial to reduce the conversion time of the sol to the gel. Those skilled in the art may adjust it as needed to achieve the solation and gelation processes.

In some examples, in the gelation step, the stirring time is between about 1-12 hours. For example, the duration time of the stirring is 1 hour, 2 hours, 3 hours, 4 hours, 5 hours, 6 hours, 7 hours, 8 hours, 9 hours, 10 hours, 11 hours, 12 hours and the like. After the sol of the soluble precursor is obtained, the sol is stirred at a selected temperature for a period of time within the above range, it may further promote the polymerization reaction process between the colloidal particles of the sol and the formation of the three-dimensional network structure. Those skilled in the art may adjust it as needed to achieve the sol-to-gel conversion process.

In some examples, in the aging step, the aging time is between about 1-3 hours. For example, the aging time is 1 hour, 2 hours, 3 hours and the like. While the aging time is within the above range, the sufficient cross-linking of the gel may be further promoted, and the three-dimensional network structure may be further stabilized. Those skilled in the art may adjust it as needed to achieve the sufficient cross-linking and stabilization processes of the gel.

In some examples, the reduction of the catalyst carrier is performed at a temperature of 200-500° C. and in the presence of hydrogen for between about 2-6 hours. For example, the reduction temperature is 200° C., 300° C., 350° C., 400° C., 450° C., 500° C. and the like. For example, the reduction time is 2 hours, 3 hours, 4 hours, 5 hours, 6 hours and the like. The hydrogen is used to reduce the catalyst carrier, and the reduction temperature and time are within the above ranges, the hydrogenation active metal in the form of the oxide may be reduced more fully, and the hydrogen molecule activation ability of the hydrogenation active metal may be further improved.

In some examples, the niobium source is selected from the group consisting of a niobium tartrate, a niobium citrate, a niobium malate, a niobium nitrate, a niobium hydrochloride, a niobium sulfate, and a combination thereof, and preferably the niobium source is the niobium tartrate. The niobium source within the scope of the present disclosure has the higher solubility relative to a commercial soluble niobium source. For example, the solubility of the niobium tartrate prepared in the present disclosure is 1.0 mol/L, while the solubility of the commercial soluble niobium source niobium oxalate or ammonium niobium oxalate is only 0.25 mol/L. The higher solubility may improve the solubility of the niobium source in water, and further promote the occurrence of the hydrolysis reaction, and thereby, it is beneficial to the preparation of the hydrodeoxygenation catalyst having the chemical formula of the present disclosure by a sol-gel method. On the other hand, the niobium source within the scope of the present disclosure may be continuously kept standing for more than three weeks after being dissolved in the water without precipitation. Compared with the commercial soluble niobium source niobium oxalate or ammonium niobium oxalate in the existing technology that may generate an apparent precipitate within 1 week, the niobium source within the scope of the present disclosure has the good stability, thereby the stability of the sol prepared therefrom is further improved. In addition, the cost of the niobium source used in the present disclosure is low, which may reduce the production cost of preparing the hydrodeoxygenation catalyst.

The hydrolyzing agent is selected from the group consisting of an acid and an alkali, and the concentration of the hydrolyzing agent is between about 0.1 mol/L and 2.0 mol/L, for example, 0.1 mol/L, 0.2 mol/L, 0.3 mol/L, 0.4 mol/L, 0.5 mol/L, 1.0 mol/L, 1.5 mol/L, and 2.0 mol/L. The acid is selected from the group consisting of a hydrochloric acid, a sulfuric acid, a nitric acid, a formic acid, an acetic acid, an oxalic acid, a citric acid and a combination thereof, and the alkali is selected from the group consisting of ammonia water, triethylamine, ethylenediamine, tetramethylethylenediamine and a combination thereof. The type and concentration of the hydrolyzing agent are within the scope of the present disclosure, which may further promote the hydrolysis process of each reactant in the solation process, as well as the polymerization and cross-linking between the colloidal particles in the gelation process, and may further reduce the sol-to-gel conversion time. Those skilled in the art may adjust it as needed to achieve the solation and gelation processes.

In some examples, the aluminum source is selected from the group consisting of an aluminum nitrate, an aluminum chloride, an aluminum acetate, and a combination thereof, and preferably the aluminum source is the aluminum nitrate. The use of the aluminum source within the scope of the present disclosure may further provide more acid sites, thereby the isomerization reaction of the alkane is further promoted. Those skilled in the art may adjust it as needed, to achieve the isomerization reaction of the alkane.

In some examples, the silicon source is selected from the group consisting of a silica sol, water glass, an ethyl orthosilicate, and a combination thereof, and preferably the silicon source is the silica sol. The use of the silicon source within the scope of the present disclosure may further provide more acid sites, thereby the isomerization reaction of the alkane is further promoted. Those skilled in the art may adjust it as needed to achieve the isomerization reaction of the alkane.

In some examples, the hydrogenation active metal source is selected from the group consisting of a nitrate, a sulfate, a chloride, an acetate and a combination thereof, preferably the hydrogenation active metal source is selected from the group consisting of a palladium nitrate, a palladium sulfate, a palladium chloride, a palladium acetate, a platinum nitrate, a platinum sulfate, a platinum chloride, a platinum acetate, a ruthenium nitrate, a nickel nitrate, a cobalt nitrate and a combination thereof, and most preferably the hydrogenation active metal source is the palladium nitrate. Those skilled in the art may adjust it as needed to achieve the hydrodeoxygenation reaction of the difurfurylacetone.

In some examples, during the preparation process of the hydrodeoxygenation catalyst, the prepared hydrodeoxygenation catalyst is subjected to further forming treatment, as to improve the mechanical strength of the hydrodeoxygenation catalyst. Herein, the forming treatment includes, but is not limited to, extruding, ball-rolling, tabletting, pelletizing and a combination thereof. Those skilled in the art may adjust it as needed to achieve the improvement in the mechanical strength of the hydrodeoxygenation catalyst.

In some examples, a method for catalyzing biomass hydrodeoxygenation to prepare an isoalkane using the hydrodeoxygenation catalyst includes: in the presence of the hydrodeoxygenation catalyst and an organic solvent, performing a hydrogenation reaction on the difurfurylacetone of the following formula to obtain the isoalkane:

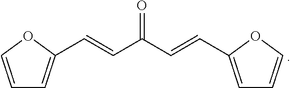 [SMILES: O=C(/C=C/c1ccco1)/C=C/c1ccco1]

Preferably, the reaction of the difurfurylacetone, the organic solvent and the hydrodeoxygenation catalyst is performed in a stainless steel high-pressure reaction kettle containing a polytetrafluoroethylene liner.

In some examples, the hydrodeoxygenation reaction is performed at a temperature of between about 150° C.-280° C., under a hydrogen pressure of between about 0.5 MPa-8 MPa, and under stirring for between about 1-36 hours. For example, the temperature of the hydrogenation reaction is 150° C., 160° C., 170° C., 180° C., 190° C., 200° C., 210° C., 220° C., 230° C., 240° C., 250° C., 260° C., 270° C., 280° C. and the like. For example, the hydrogen pressure is 0.5 MPa, 0.6 MPa, 0.7 MPa, 0.8 MPa, 0.9 MPa, 1.0 MPa, 2.0 MPa, 3.0 MPa, 4.0 MPa, 5.0 MPa, 6.0 MPa, 7.0 MPa, 8.0 MPa and the like. For example, the reaction is performed for 1 hour, 2 hours, 3 hours, 4 hours, 5 hours, 6 hours, 7 hours, 8 hours, 9 hours, 10 hours, 15 hours, 20 hours, 25 hours, 30 hours, 32 hours, 34 hours, 36 hours and the like. Those skilled in the art may adjust it as needed to achieve the hydrodeoxygenation reaction of the difurfurylacetone catalyzed by the hydrodeoxygenation catalyst.

In some examples, the organic solvent in the hydrogenation reaction is selected from the group consisting of a cyclohexane, a bio-jet fuel and other conventional jet fuels, and preferably the organic solvent is the cyclohexane. Based on the total weight of the difurfurylacetone and the organic solvent in the hydrogenation reaction, the weight percent of the difurfurylacetone is between about 10 wt %-30 wt %, for example, 10 wt %, 15 wt %, 20 wt %, 25 wt %, 30 wt % and the like. The weight ratio of the difurfurylacetone to the hydrodeoxygenation catalyst (the ratio of the weight of the difurfurylacetone to the weight of the hydrodeoxygenation catalyst) is (2-20): 1, for example, 2:1, 3:1, 4:1, 5:1, 6:1, 7:1, 8:1, 9:1, 10:1, 12:1, 14:1, 16:1, 18:1, 20:1 and the like. Those skilled in the art may adjust it as needed to achieve the hydrodeoxygenation reaction of the difurfurylacetone catalyzed by the hydrodeoxygenation catalyst.

The present application is further described in detail below with reference to specific examples, and these examples should not be construed as limiting the scope of protection claimed by the present application.

### Catalyst Preparation and Use Examples:

In the following test examples and test contrast examples, the selectivity of the isoalkane refers to a proportion of the isoalkane accounting for the total alkane in the product, and it may be calculated by the following formula: selectivity of isoalkane (%)=weight of isoalkane in product/weight of total alkane in product.

### Preparation Example 1

0.5 g of an aluminum nitrate, and 4 g of a silica sol (the silicon dioxide concentration was 11% w/w) were weighed, 0.5 mL of palladium nitrate solution (0.01 g/mL) was measured, and 0.5 g of citric acid was added to 20 mL of deionized water, and they were mixed uniformly; 10 mL of niobium tartrate solution (0.7 mol/L) was measured and added to the above solution to form a sol. Stirring was kept at a constant temperature of 35° C. in a water bath for 2 hours until a gel was formed. Stand the gel for 1 hour. Then the gel was dehydrated and dried under a condition of 100° C., and a dried sample was heated to 500° C. at a heating rate of 1° C./min in a muffle furnace, and was naturally cooled to a room temperature after being roasted for 300 min, it was reduced at 200° C. with hydrogen for 2 hours before use, and finally a catalyst 1 was obtained. By X-ray fluorescence (XRF) measurement, a carrier chemical formula of the catalyst 1 was ((Nb₂O₅)ₓ·(Al₂O₃)_{y}·(SiO₂)_{z}, wherein x:y:z was 0.28:0.07:0.65), and the content of an active metal Pd was 0.4 wt %.

### Preparation Example 2

A preparation method was the same as that of Preparation Example 1, except that the addition amount of the aluminum nitrate was 0.1 g, the addition amount of the silica sol (the silicon dioxide concentration is 11% w/w) was 6 g, 0.3 mL of the palladium nitrate solution (0.01 g/mL) was measured, the addition amount of the niobium tartrate solution (0.7 mol/L) was 2 mL, conversion of sol to gel was performed under the condition of 60° C. in a water bath, and stand the gel for 3 hours. Finally, a catalyst 2 was obtained. By XRF measurement, a chemical formula of a carrier in the catalyst 2 was ((Nb₂O₅)ₓ·(Al₂O₃)_{y}·(SiO₂)_{z}, wherein x:y:z was 0.05:0.02:0.93), and the content of an active metal Pd was 0.48 wt %.

### Preparation Example 3

A preparation method was the same as that of Preparation Example 1, except that the addition amount of the aluminum nitrate was 0.25 g, the addition amount of the silica sol (the silicon dioxide concentration was 11% w/w) was 5 g, 0.42 mL of the palladium nitrate solution (0.01 g/mL) was measured, and the addition amount of the niobium tartrate solution (0.7 mol/L) was 6.7 mL, conversion of sol to gel was performed at room temperature, and stand the gel for 3 hours. Finally, a catalyst 3 was obtained. By XRF measurement, a chemical formula of a carrier in the catalyst 3 was ((Nb₂O₅)ₓ·(Al₂O₃)_{y}·(SiO₂)_{z}, wherein x:y:z was 0.21:0.05:0.74), and the content of an active metal Pd was 0.4 wt %.

### Preparation Contrast Example 1

A preparation method was the same as that of Preparation Example 1, except that the aluminum nitrate was not added. Finally, a catalyst A (without Al) was obtained. By XRF measurement, a chemical formula of the catalyst A was ((Nb₂O₅)ₓ·(SiO₂)_{z}, wherein x: z was 0.28:0.65), and the content of an active metal Pd was 0.44 wt %.

### Preparation Contrast Example 2

A preparation method was the same as that of Preparation Example 1, except that the silica sol was not added. Finally, a catalyst B (without Si) was obtained. By XRF measurement, a chemical formula of the catalyst B was ((Nb₂O₅)ₓ·(Al₂O₃)_{y}, wherein x: y was 0.28:0.07), and the content of an active metal Pd was 0.57 wt %.

### Preparation Contrast Example 3

A preparation method was the same as that of Preparation Example 1, except that the niobium tartrate was not added. Finally, a catalyst C (without Nb) was obtained. By XRF measurement, a chemical formula of the catalyst C was ((Al₂O₃)_{y}·(SiO₂)_{z}, wherein y:z was 0.07:0.65), and the content of an active metal Pd was 1.1 wt %.

### Preparation Contrast Example 4

A preparation method was the same as that of Preparation Example 1, except that 3.0 g of the aluminum nitrate was added, and 0.7 mL of the palladium nitrate solution (0.01 g/mL) was measured. Finally, a catalyst D was obtained. By XRF measurement, a chemical formula of the catalyst D was ((Nb₂O₅)ₓ·(Al₂O₃)_{y}·(SiO₂)_{z}, wherein x:y:z was 0.19:0.36:0.45), and the content of an active metal Pd was 0.4 wt %.

### Preparation Contrast Example 5

A preparation method was the same as that of Preparation Example 1, except that 2.2 g of the silica sol was added. Finally, a catalyst E was obtained. By XRF measurement, a chemical formula of the catalyst E was ((Nb₂O₅)ₓ·(Al₂O₃)_{y}·(SiO₂)_{z}, wherein x:y:z was 0.55:0.13:0.32), and the content of an active metal Pd was 0.41 wt %.

### Preparation Contrast Example 6

A preparation method was the same as that of Preparation Example 1, except that 40 mL of the niobium tartrate solution (0.5 mol/L) was added, and 0.8 mL of the palladium nitrate solution (0.01 g/mL) was measured. Finally, a catalyst F was obtained. By XRF measurement, a chemical formula of the catalyst F obtained was ((Nb₂O₅)ₓ·(Al₂O₃)_{y}·(SiO₂)_{z}, wherein x:y:z was 0.61:0.03:0.36), and the content of an active metal Pd was 0.42 wt %.

The preparation examples and the preparation contrast examples are summarized in Tables 1A-1C.

TABLE 1A

| Example | Preparation | Preparation | Preparation |
|---|---|---|---|
| Number | Example 1 | Example 2 | Example 3 |
| Catalyst | 1 | 2 | 3 |
| Number | | | |
| Composition | (Nb₂O₅)_{0.28}* | (Nb₂O₅)_{0.05}* | (Nb₂O₅)_{0.21}* |
| | (Al₂O₃)_{0.07}* | (Al₂O₃)_{0.02}* | (Al₂O₃)_{0.05}* |
| | (SiO₂)_{0.65} | (SiO₂)_{0.93} | (SiO₂)_{0.74} |

TABLE 1B

| | Preparation | Preparation | Preparation |
|---|---|---|---|
| Example | Contrast | Contrast | Contrast |
| Number | Example 1 | Example 2 | Example 3 |
| Catalyst | A | B | C |
| Number | | | |
| Composition | (Nb₂O₅)_{0.28}* | (Nb₂O₅)_{0.28}* | (Al₂O₃)_{0.07}* |
| | (SiO₂)_{0.65} | (Al₂O₃)_{0.07} | (SiO₂)_{0.65} |

TABLE 1C

| | Preparation | Preparation | Preparation |
|---|---|---|---|
| Example | Contrast | Contrast | Contrast |
| Number | Example 4 | Example 5 | Example 6 |
| Catalyst | D | E | F |
| Number | | | |
| Composition | (Nb₂O₅)_{0.19}* | (Nb₂O₅)_{0.55}* | (Nb₂O₅)_{0.61}* |
| | (Al₂O₃)_{0.36}* | (Al₂O₃)_{0.13}* | (Al₂O₃)_{0.03}* |
| | (SiO₂)_{0.45} | (SiO₂)_{0.32} | (SiO₂)_{0.36} |

### Test Example 4

0.1 g of a catalyst 1, 1.0 g of a difurfurylacetone, and 4 g of a cyclohexane were taken and placed in a 30 mL stainless steel high-pressure reaction kettle with a polytetrafluoroethylene liner, and after leak detection, hydrogen was fed and maintained at 3.5 MPa. Then, at a reaction temperature of 180° C., it was stirred at a high speed for 12 hours to obtain a long-chain alkane. The content of the long-chain alkane in a reaction product was measured by a gas chromatography (GC). As a result, the conversion rate of the difurfurylacetone was 100%, the yield of C₁₀₊ alkane (C₁₀-C₁₃ total alkane) was 91%, and the selectivity of an isoalkane was 40%.

### Test Example 5

A test method was the same as that of Test Example 4, except that the catalyst 1 was replaced with a catalyst 2. The content of a long-chain alkane in a reaction product was measured by GC. As a result, the conversion rate of a difurfurylacetone was 99%, the yield of C₁₀₊ alkane (C₁₀-C₁₃ total alkane) was 89%, and the selectivity of an isoalkane was 38%.

### Test Example 6

A test method was the same as that of Test Example 4, except that the catalyst 1 was replaced with a catalyst 3. The content of a long-chain alkane in a reaction product was measured by GC. As a result, the conversion rate of a difurfurylacetone was 100%, the yield of C₁₀₊ alkane (C₁₁-C₁₃ alkane) was 90%, and the selectivity of an isoalkane was 39%.

### Test Contrast Example 7

A test method was the same as that of Test Example 4, except that the catalyst 1 was replaced with a catalyst A. The content of a long-chain alkane in a reaction product was measured by GC. As a result, the conversion rate of a difurfurylacetone was 100%, the yield of C₁₀₊ alkane (C₁₁-C₁₃ alkane) was 88%, and the selectivity of an isoalkane was 12%.

### Test Contrast Example 8

A test method was the same as that of Test Example 4, except that the catalyst 1 was replaced with a catalyst B. The content of a long-chain alkane in a reaction product was measured by GC. As a result, the conversion rate of a difurfurylacetone was 100%, the yield of C₁₀₊ alkane (an alkane with a carbon number greater than 10) was 80%, and the selectivity of an isoalkane was 35%.

### Test Contrast Example 9

A test method was the same as that of Test Example 4, except that the catalyst 1 was replaced with a catalyst C. The content of a long-chain alkane in a reaction product was measured by GC. As a result, the conversion rate of a difurfurylacetone was 100%, the yield of C₁₀₊ alkane (an alkane with a carbon number greater than 10) was 56%, and the selectivity of an isoalkane was 8%.

### Test Contrast Example 10

A test method was the same as that of Test Example 4, except that the catalyst 1 was replaced with a catalyst D. The content of a long-chain alkane in a reaction product was measured by GC. As a result, the conversion rate of a difurfurylacetone was 100%, the yield of C₁₀₊ alkane (an alkane with a carbon number greater than 10) was 81%, and the selectivity of an isoalkane was 35%.

### Test Contrast Example 11

A test method was the same as that of Test Example 4, except that the catalyst 1 was replaced with a catalyst E. The content of a long-chain alkane in a reaction product was measured by GC. As a result, the conversion rate of a difurfurylacetone was 100%, the yield of C₁₀₊ alkane (C₁₁-C₁₃ alkane) was 81%, and the selectivity of an isoalkane was 33%.

### Test Contrast Example 12

A test method was the same as that of Test Example 4, except that the catalyst 1 was replaced with a catalyst F. The content of a long-chain alkane in a reaction product was measured by GC. As a result, the conversion rate of a difurfurylacetone was 100%, the yield of C₁₀₊ alkane (C₁₁-C₁₃ alkane) was 89%, and the selectivity of an isoalkane was 5%.

The test examples and test contrast examples are summarized in Tables 2A-2C.

TABLE 2A

| | Test | Test | Test |
|---|---|---|---|
| Example Number | Example 4 | Example 5 | Example 6 |
| Catalyst Number | 1 | 2 | 3 |
| Conversion Rate of | 100% | 99% | 100% |
| Difurfurylacetone | | | |
| Yield of C₁₀₊ Alkane | 91% | 89% | 90% |
| Selectivity of | 40% | 38% | 39% |
| Isoalkane | | | |

TABLE 2B

| | Test Contrast | Test Contrast | Test Contrast |
|---|---|---|---|
| Example Number | Example 7 | Example 8 | Example 9 |
| Catalyst Number | A | B | C |
| Conversion Rate of | 100% | 100% | 100% |
| Difurfurylacetone | | | |
| Yield of C₁₀₊ Alkane | 88% | 80% | 56% |
| Selectivity of | 12% | 35% | 8% |
| Isoalkane | | | |

TABLE 2C

| | Test Contrast | Test Contrast | Test Contrast |
|---|---|---|---|
| Example Number | Example 10 | Example 11 | Example 12 |
| Catalyst Number | D | E | F |
| Conversion Rate of | 100% | 100% | 100% |
| Difurfurylacetone | | | |
| Yield of C₁₀₊ Alkane | 81% | 81% | 89% |
| Selectivity of | 35% | 33% | 5% |
| Isoalkane | | | |

From the above descriptions, it may be seen that the above examples of the present disclosure achieve the following technical effects. The present disclosure greatly improves the yield and selectivity of the long-chain alkane in the process of preparing the bio-jet fuel component oil from the biomass. Compared with Test contrast examples 7-12, the C₁₀₊ alkane yield and isoalkane selectivity of Test examples 4-6 are significantly higher, namely the C₁₀₊ alkane yield of the hydrodeoxygenation catalyst of the present disclosure may reach about 90%, in which the isoalkane selectivity may reach nearly 40%. It is achieved that the biomass is effectively converted into the bio-jet fuel component oil in the lower industrial cost and mild reaction conditions.

Further, the disclosure comprises examples according to the following clauses:
Clause 1. A hydrodeoxygenation catalyst, wherein the hydrodeoxygenation catalyst comprises a hydrogenation active component and a catalyst carrier, wherein the hydrogenation active component comprises one or more hydrogenation active metals, and the catalyst carrier is a solid solution composite oxide containing Nb, Al, Si, and O elements, and the catalyst carrier is represented by a chemical formula (Nb₂O₅)ₓ·(Al₂O₃)_{y}·(SiO₂)_{z}, wherein 0.01≤x≤0.3, 0.01≤y≤0.1, and 0.6≤z≤0.98.
Clause 2. The hydrodeoxygenation catalyst according to Clause 1, wherein the one or more hydrogenation active metals comprise Pt, Pd, Rh, Ru, Ni, Co, Cu, or a combination thereof.
Clause 3. The hydrodeoxygenation catalyst according to Clause 1 or 2, wherein based on the total weight of the hydrodeoxygenation catalyst, the loading amount of the hydrogenation active metal is between about 0.4 wt %-10 wt %.
Clause 4. The hydrodeoxygenation catalyst according to any of Clauses 1-3, wherein Nb₂O₅, Al₂O₃, and SiO₂ in the hydrodeoxygenation catalyst exist in an amorphous form.
Clause 5. A preparation method for the hydrodeoxygenation catalyst according to any of Clauses 1-4, wherein the method comprises: a solation step: mixing a hydrogenation active metal source with a niobium source, an aluminum source and a silicon source of which the molar ratio is (0.01-0.3):(0.01-0.1):(0.6-0.98) and dissolving in water, to obtain a sol of a soluble precursor; a gelation step: stirring the sol at a temperature from a room temperature to 60° C., to obtain a gel; and an aging step: standing the gel for aging, to obtain an aged material; and drying, calcining and reducing the aged material, to obtain the hydrodeoxygenation catalyst.
Clause 6. The method according to Clause 5, wherein in the solation step, the mixing of the niobium source, the aluminum source, the silicon source and the hydrogenation active metal source is performed in the presence of a hydrolyzing agent.
Clause 7. The method according to Clause 6, wherein the hydrolyzing agent is selected from the group consisting of an acid and an alkali.
Clause 8. The method according to Clause 7, wherein the concentration of the hydrolyzing agent is between about 0.1 mol/L-2.0 mol/L,
Clause 9. The method according to any of Clauses 5-8, wherein in the gelation step, the stirring time is between about 1-12 hrs.
Clause 10. The method according to any of Clauses 5-9, wherein in the aging step, the aging time is between about 1-3 hrs.
Clause 11. The method according to any of Clauses 5-10, wherein the reduction is performed at a temperature of between about 200-500° C. and in the presence of hydrogen for between about 2-6 hrs.
Clause 12. The method according to any of Clauses 5-11, wherein the niobium source is selected from the group consisting of a niobium tartrate, a niobium citrate, a niobium malate, a niobium nitrate, a niobium hydrochloride, a niobium sulfate, and a combination thereof.
Clause 13. The method according to any of Clauses 5-12, wherein, the aluminum source is selected from the group consisting of an aluminum nitrate, an aluminum chloride, an aluminum acetate, and a combination thereof, the silicon source is selected from the group consisting of a silica sol, water glass, an ethyl orthosilicate and a combination thereof, and preferably the silicon source is the silica sol; and the hydrogenation active metal source is selected from the group consisting of a nitrate, a sulfate, a chloride, an acetate and a combination thereof, and preferably the hydrogenation active metal source is selected from the group consisting of a palladium nitrate, a palladium sulfate, a palladium chloride, a palladium acetate, a platinum nitrate, a platinum sulfate, a platinum chloride, a platinum acetate, a ruthenium nitrate, a nickel nitrate, a cobalt nitrate, and a combination thereof.
Clause 14. A method for preparing an isoalkane using the hydrodeoxygenation catalyst according to any of Clauses 1-4, wherein the method comprises: in the presence of the hydrodeoxygenation catalyst and an organic solvent, performing a hydrogenation reaction on the difurfurylacetone of the following formula to obtain the isoalkane:

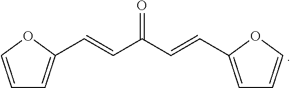 [SMILES: O=C(/C=C/c1ccco1)/C=C/c1ccco1]

Clause 15. The method according to Clause 14, wherein the hydrodeoxygenation reaction is performed at a temperature of between about 150° C.-280° C., under a hydrogen pressure of between about 0.5 MPa-8 MPa, and under stirring for between about 1 hr-36 hrs.
Clause 16. The method according to Clause 14 or 15, wherein the organic solvent is a cyclohexane, and based on the total weight of the difurfurylacetone and the organic solvent, the weight percent of the difurfurylacetone is between about 10 wt %-30 wt %.
Clause 17. The method according to any of Clauses 14-16, wherein the weight ratio of the difurfurylacetone to the hydrodeoxygenation catalyst is (2-20): 1.

The terminology used herein is for the purpose of describing particular examples only and is not intended to be limiting of examples. As used herein, the singular forms “a,” “an,” and “the” are intended to include the plural forms as well, unless the context clearly indicates otherwise. It will be further understood that the terms “comprises,” “comprising,” “includes,” and/or “including,” when used in this specification, specify the presence of stated features, integers, steps, operations, elements, and/or components, but do not preclude the presence or addition of one or more other features, integers, steps, operations, elements, components, and/or groups thereof.

It should be noted that terms “first”, “second” and the like in the description and claims of the present application are used to distinguish similar objects, and are not necessarily used to describe a specific sequence or a precedence order. It should be understood that the terms so used are interchangeable under appropriate circumstances, such that the examples of the present application described here may, for example, be implemented in a sequence other than those described here.

The terms “comprising”, “comprise” and “comprises” herein are intended to be optionally substitutable with the terms “consisting essentially of”, “consist essentially of”, “consists essentially of”, “consisting of”, “consist of” and “consists of”, respectively, in every instance.

Unless otherwise indicated, approximating language, such as “generally,” “substantially,” and “about,” as used herein indicates that the term so modified may apply to only an approximate degree, as would be recognized by one of ordinary skill in the art, rather than to an absolute or perfect degree. Accordingly, a value modified by a term or terms such as “about,” “approximately,” and “substantially” is not to be limited to the precise value specified. For example, the approximating language may correspond to the precision of an instrument for measuring the value.

The above are only preferred examples of the present disclosure, and are not intended to limit the present disclosure. For those skilled in the art, the present disclosure may have various modifications and changes. Any modifications, equivalent replacements, improvements and the like made within the spirit and principle of the present disclosure shall be included within the scope of protection of the present disclosure.

1-17. (canceled)

18. A hydrodeoxygenation catalyst, wherein the hydrodeoxygenation catalyst comprises:
a hydrogenation active component, wherein the hydrogenation active component comprises one or more hydrogenation active metals; and
a catalyst carrier including a solid solution composite oxide represented by a chemical formula (Nb₂O₅)ₓ·(Al₂O₃)_{y}·(SiO₂)_{z}, wherein 0.01≤x≤0.3, 0.01≤y≤0.1, and 0.6≤z≤0.98.

19. The hydrodeoxygenation catalyst of claim 18, wherein the one or more hydrogenation active metals comprise Pt, Pd, Rh, Ru, Ni, Co, Cu, or a combination thereof.

20. The hydrodeoxygenation catalyst according to claim 19, wherein, based on a total weight of the hydrodeoxygenation catalyst, a loading amount of the one or more hydrogenation active metals is between about 0.4 wt % and about 10 wt %.

21. The hydrodeoxygenation catalyst of claim 18, wherein Nb₂O₅, Al₂O₃, and SiO₂ in the hydrodeoxygenation catalyst exist in an amorphous form.

22. The hydrodeoxygenation catalyst of claim 18, wherein the hydrogenation active component and the catalyst carrier are prepared by:
solation, in water, of a hydrogenation active metal source with a niobium source, an aluminum source, and a silicon source to obtain a sol;
gelation of the sol to obtain a gel;
age the gel for a time period to obtain an aged material; and
process the aged material.

23. The hydrodeoxygenation catalyst of claim 22, wherein said process the aged material includes:
dry the aged material;
calcinate the aged material; and
reduce the aged material with hydrogen.

24. The hydrodeoxygenation catalyst of claim 22, wherein the solation includes addition of a hydrolyzing agent to the water.

25. A preparation method for a hydrodeoxygenation catalyst, wherein the method comprises:
mixing, in water, a hydrogenation active metal source with a niobium source, an aluminum source, and a silicon source to obtain a sol, wherein a molar ratio of the niobium source to the aluminum source to the silicon source is (0.01 to 0.3):(0.01 to 0.1):(0.6 to 0.98);
stirring the sol to obtain a gel, wherein a temperature of the sol is less than 60° C.;
standing the gel for a period of time to obtain an aged material; and
processing the aged material to obtain the hydrodeoxygenation catalyst, wherein hydrodeoxygenation catalyst includes a hydrogenation active component and a catalyst carrier, wherein the hydrogenation active component comprises one or more hydrogenation active metals, wherein the catalyst carrier is represented by a chemical formula (Nb₂O₅)ₓ·(Al₂O₃)_{y}·(SiO₂)_{z}, and wherein 0.01≤x≤0.3, 0.01≤y≤0.1, and 0.6≤z≤0.98.

26. The method of claim 25, wherein said mixing is performed in presence of a hydrolyzing agent.

27. The method of claim 26, wherein the hydrolyzing agent is selected from the group consisting of an acid and an alkali.

28. The method of claim 26, wherein a concentration of the hydrolyzing agent is between about 0.1 mol/L and 2.0 mol/L.

29. The method of claim 25, wherein said stirring is performed for a first time between about an hour and about 12 hours.

30. The method of claim 25, wherein the period of time is between about an hour and about 3 hours.

31. The method of claim 25, wherein said processing includes:
drying the aged material;
calcining the aged material; and
reducing the aged material at a temperature below about 500° C. in presence of hydrogen for a second time period between about 2 hours to about 6 hours.

32. The method of claim 25, wherein the niobium source is selected from the group consisting of niobium tartrate, niobium citrate, niobium malate, niobium nitrate, niobium hydrochloride, niobium sulfate, or combinations thereof.

33. The method of claim 25, wherein:
the aluminum source is selected from a first group consisting of aluminum nitrate, aluminum chloride, aluminum acetate, or combinations thereof;
the silicon source is selected from a second group consisting of silica sol, water glass, an ethyl orthosilicate, or combinations thereof; and
the hydrogenation active metal source is a nitrate, a sulfate, a chloride, an acetate, or combinations thereof, of one or more of a third group consisting of Pt, Pd, Rh, Ru, Ni, Co, and Cu.

34. A method to prepare an isoalkane, the method comprising:
obtaining a hydrodeoxygenation catalyst, wherein the hydrodeoxygenation catalyst includes a hydrogenation active component and a catalyst carrier, wherein the hydrogenation active component comprises one or more hydrogenation active metals, wherein the catalyst carrier is represented by a chemical formula (Nb₂O₅)ₓ·(Al₂O₃)_{y}·(SiO₂)_{z}, and wherein 0.01≤x≤0.3, 0.01≤y≤0.1, and 0.6≤z≤0; and
in a reaction vessel and in presence of the hydrodeoxygenation catalyst and an organic solvent, performing a hydrodeoxygenation reaction on difurfurylacetone to produce the isoalkane.

35. The method of claim 34, wherein the hydrodeoxygenation reaction is performed within a temperature range between about 150° C. to about 280° C., under a hydrogen pressure range between about 0.5 MPa and about 8 MPa, and under stirring for about an hour to about 36 hours.

36. The method of claim 34, wherein the organic solvent is a cyclohexane, and based on a total weight of the difurfurylacetone and the organic solvent, a weight percent of the difurfurylacetone is between about 10 wt % and 30 wt %.

37. The method of claim 34, wherein a weight ratio of the difurfurylacetone to the hydrodeoxygenation catalyst prior to the hydrodeoxygenation reaction is in a range from about 2:1 to about 20:1.
